(19) United States
(12) Patent Application Publication
(10) Pub. No.: US 2026/0306414 A1
(43) Pub. Date: Oct. 8, 2026

(54) Composition and Method for Topical Trans-spinal Delivery of Nucleic Acids and Small Molecules

(71) Applicant: 3P Biotechnologies, Inc., PROSPECT, KY (US)

(72) Inventors: Ramesh Gupta, Prospect, KY (US); Wendy Spencer, Lexington, KY (US)

(21) Appl. No.: 19/213,040

(22) Filed: May 20, 2025

Related U.S. Application Data
(60) Provisional application No. 63/651,648, filed on May 24, 2024.
(60) Provisional application No. 63/651,679, filed on May 24, 2024.

(51) Int. Cl. A61K 9/50; A61K 9/00; A61K 31/12; A61K 35/20; A61P 25/28; C12N 15/113

(52) U.S. Cl. CPC A61K 9/5068; A61K 9/0014; A61K 31/12; A61K 35/20; A61P 25/28; C12N 15/113; C12N 2310/11; C12N 2310/14

(57) ABSTRACT

A method to mitigate neuroinflammatory diseases is described. The method involves the administration of at least one therapeutic agent via a topical trans-spinal route The therapeutic agent is a nucleic acid, such as siRNA, anti-sense oligomer (ASO), plasmid DNA, or a curcuminoid, such as curcumin, demethoxycurcumin, bisdemethoxycurcumin, or any combination thereof, delivered using an EPM-nucleic acid complex or an ExoCUR complex. The EPM-nucleic acid complex or the ExoCUR complex can be blended with a pharmaceutically acceptable vehicle, carrier, or excipient.

### CROSS-REFERENCE TO PRIOR APPLICATIONS

The present application claims priority to U.S. 63/651,648, filed 24 May 2024, currently pending, which is incorporated by reference in its entirety, and to U.S. 63/651,679, filed 24 May 2024, currently pending, which is incorporated by reference in its entirety.

### FEDERALLY SPONSORED RESEARCH AND DEVELOPMENT

Not applicable.

### JOINT RESEARCH AGREEMENT

Not applicable.

### REFERENCE TO A “SEQUENCE LISTING”, A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC

Not applicable.

### STATEMENT REGARDING PRIOR DISCLOSURES BY AN INVENTOR OR JOINT INVENTOR

Not applicable.

### BACKGROUND OF THE INVENTION

### (1) Field of the Invention

The presently disclosed subject matter relates to a method for the topical delivery of therapeutic agents, and particularly for delivery of therapeutic agents to the brain and/or central nervous system.

### (2) Description of Related Art

Inflammation is a protective response of the host to infections and tissue injury characterized by a complex series of reactions involving immune cells, blood vessels and molecular mediators. Under normal circumstances, inflammation is beneficial and characterized by a restricted and transient upregulation of inflammatory activity. However, dysregulated inflammation can cause long-lasting tissue damage and contributes to both acute and chronic inflammatory diseases. Chronic inflammation is recognized as the most significant cause of death worldwide accounting for >50% of all deaths for a broad range of inflammation-mediated diseases including neurodegenerative diseases. Neurodegenerative disorders affect over 40 million people worldwide and most, if not all, are mediated by inflammation.

The transcription factor family NFκB, a fundamental regulator of the inflammatory response, is emerging as central and pivotal player in exacerbating neuroinflammatory diseases. Finding ways to inhibit the NFκB signaling pathway is an attractive target for drug development. Memantine has been shown to block NFκB activity and NFκB-dependent adhesion molecules in the human brain. NSAIDs has been shown to inhibit NFκB-induced expression of BACE1, decrease Alzheimer's Disease (AD)-like pathology in mice, and inhibit the IKK kinase complex preventing NFκB activation, reducing the risk of developing AD. However, the efficacy of memantine and of NSAIDs as AD therapeutics has not been realized as only 1-2% of oral NSAID dosing reaches the brain.

RNAi mediated by siRNA is a type of gene therapy that targets abnormal gene expression by directing degradation of overly abundant mRNA. Using siRNA as a therapeutic has several attractive features: 1) any gene can be targeted; 2) siRNA acts in picomolar range; and 3) infrequent dosing achieves therapeutic effects. The mechanism of siRNA knockdown of gene expression can be saturated and is thus unlikely to achieve 100% knockdown, making it an ideal therapeutic for the overexpression of normal proteins that lead to pathology. However, siRNA delivery to the desired target remains a challenge.

Alzheimer's disease (AD), characterized by progressive loss of memory and cognitive function, is the most prevalent cause of dementia worldwide. Estimates indicate that 35 million people are currently afflicted with this age-related chronic neurodegenerative disease and by 2050 this number is expected to triple. Three core features are generally recognized as key contributors to the pathogenesis of AD: 1) formation of Aβ plaques, generated by BACE1 (β-amyloid precursor protein cleaving enzyme)-catalyzed cleavage of APP (amyloid precursor protein), 2) generation of neurofibrillary tangles (NFTs) arising from hyperphosphorylation of the tau microtubular-associated protein (pTau), and 3) sustained oxidative stress and neuroinflammation. Aβ plaques and NFTs are observed prior to AD pathology in clinical populations and preclinical animal models and the risk of AD dementia increases with progression from normal cognition and absence of Aβ plaques to early neurodegeneration and subsequently mild cognitive impairment. Although data is limited, it suggests that subtle cognitive decline with AD biomarker positivity could be a determinant for clinical treatment.

One emerging candidate for treatment of AD is curcuminoids, originally identified from the perennial herb Curcuma longa L (turmeric). Although the exploration of curcuminoids for the treatment of AD is relatively recent, its medicinal properties are well known. Curcuminoids were first implicated as protective against AD following a population-based study showing a 4.4-fold higher incidence of AD in US populations of 70-79-year-olds compared to those in India, attributed to the higher consumption of turmeric containing curcuminoids in Indian populations. Curcuminoids interfere with Aβ biogenesis by intercalating among peptide chains. They also aid in the clearance of Aβ deposits preventing the formation of Aβ aggregates by modulating expression of genes critical for plaque formation, both nuclear and mitochondrial. Curcuminoids also prevent the formation of NFTs by inhibiting pTau, aggregation of pTau and enhancing microtubule instability. BDMC, an active but minor derivative of curcumin, can improve cognition, decrease neuronal atrophy and reduce the deposition of Aβ plaques and oxidative stress in the brains of AD mice and these neuroprotective effects are attributed to the upregulation of SIRT1, a regulator of oxidative stress.

In addition to low oral bioavailability, multiple factors including low penetration in brain and targeting efficacy have limited the clinical applications of curcuminoids for AD. Of special consideration for AD therapeutics is penetration of the blood brain barrier (BBB). The BBB is formed from polarized endothelial cells and contains a large number of transporters which prevents nearly all macromolecules and >98% of small molecules from passively penetrating the central nervous system (CNS). Carrier-, receptor- and adsorptive-mediated pathways are generally required for BBB penetration. Like the BBB, the blood-cerebrospinal fluid barrier (BCSFB) found in the choroid plexus regulates the permeability of molecules from the blood into the CSF. Some of these permeability limitations can be circumvented using specific targeted nanocarriers and have been the subject of several recent reviews.

Exosomes, natural bilayer-bound nanoparticles (30-150 nm), are an emerging vector for nucleic acid delivery and for small molecule delivery. Exosomes are non-toxic, non-immunogenic and render higher efficacy as compared to nano-carriers, such as liposomes, due to longer blood-circulation due to the presence of anti-phagocytic CD47. It has been found that milk and colostrum exosomes, which are well tolerated by the human body, can effectively deliver small molecules, and a novel nanoplatform for delivery of siRNA based on the ionic interaction of the exosomes with PEI has been disclosed in U.S. Pat. No. 17,425,928 and PCT/US2020/015259, which are incorporated herein in their entireties by reference. Exosomes derived from mammalian cell culture have been loaded with both siRNA and miRNA, targeting regulation of gene expression of BACE1 in AD, EGFR in breast cancer, and mutant KRAS in pancreatic cancer. In addition, exosomes can easily cross the BBB. An early study on AD therapeutics demonstrated that exosomes isolated from cell culture can be loaded with siRNA targeting expression of BACE1. A more recent study supports the use of exosomes to deliver quercetin to improve bioavailability, target the brain and improve cognition. Exosomes have also shown intrinsic value as prospective AD therapeutics by effectuating key AD biomarkers and mediators in the absence of payload.

It would be beneficial if an effective delivery system for brain targeting for nucleic acids and small molecules could be developed. It would further be beneficial if an effective delivery system for a topical delivery of therapeutic agents, and particularly for delivery of therapeutic agents to the brain and/or central nervous system, could be developed.

### SUMMARY OF THE PRESENT INVENTION

The present development is a pharmaceutical composition for delivering nucleic acids and/or small molecules to the brain wherein the nucleic acids and/or small molecules are administered via a topical trans-spinal route. In some embodiments, the nucleic acid or small molecule being delivered is siRNA, anti-sense oligomer (ASO), plasmid DNA, or curcuminoids. In some embodiments, the pharmaceutical composition comprises an exosome—cationic matrix or an exosome—curcuminoid complex. In some embodiments, the pharmaceutical composition comprises an exosome—cationic matrix or an exosome—curcuminoid complex comprising a milk exosome or a colostrum exosome or a combination thereof.

In some embodiments, the nucleic acids and/or curcuminoids are delivered to the brain. In some embodiments, the nucleic acids or curcuminoids are delivered to the brain at a concentration effective to reduce the inflammatory regulator NFκB or the neuroinflammation to baseline levels.

In some embodiments, the exosome—cationic matrix complex or the exosome—curcuminoid complex forms part of a pharmaceutical composition, wherein the exosome—cationic matrix complex or the exosome—curcuminoid complex is combined with a pharmaceutically-acceptable vehicle, carrier, or excipient. In some embodiments, the pharmaceutical composition comprises an exosome—cationic matrix or an exosome—curcuminoid complex administered via a topical trans-spinal route.

In some embodiments, the pharmaceutical composition comprises an exosome—cationic matrix and siRNA. In some embodiments, the pharmaceutical composition comprises an exosome-cationic matrix and siRNA and folic acid. In some embodiments, the pharmaceutical composition comprises an exosome—cationic matrix and siRNA and rabies virus glycoprotein. In some embodiments, the pharmaceutical composition comprises an exosome—cationic matrix and siRNA and rabies virus glycoprotein and folic acid.

In some embodiments, the siRNA is siNFκB (siRNA targeting against NFκB expression), siBACE1, siSTAT3 or siEGFR. In some embodiments, the siRNA is siNFκB, siBACE1, siSTAT3 or siEGFR delivered to the brain. In some embodiments, the siRNA is siNFκB delivered to the brain at a concentration effective to reduce the inflammatory regulator NFκB to baseline levels. In some embodiments, the siRNA is siBACE1 delivered to the brain at a concentration effective to downregulate the overexpression of the target BACE1 (beta-site amyloid precursor protein cleaving enzyme 1) associated with AD pathology. In some embodiments, the siRNA is siSTAT3 delivered to the brain at a concentration effective to knockdown STAT3 associated with the progression of glioblastomas. Signal transducer and activator of transcription 3 (STAT3) is a tumor-promoting factor that increases the progression of gliobastoma. In some embodiments, the siRNA is siEGFR delivered to the brain at a concentration effective to downregulate the amplification and overexpression of the target epidermal growth factor receptor (EGFR) associated with glioblastomas.

In some embodiments, the pharmaceutical composition comprises an exosome—cationic matrix and ASO. In some embodiments, the pharmaceutical composition comprises an exosome—cationic matrix and ASO and folic acid. In some embodiments, the pharmaceutical composition comprises an exosome—cationic matrix and ASO and rabies virus glycoprotein. In some embodiments, the pharmaceutical composition comprises an exosome—cationic matrix and ASO and rabies virus glycoprotein and folic acid. In some embodiments, the ASO is ASO-α-synuclein. In some embodiments, the ASO is ASO-α-synuclein delivered to the brain at a concentration effective to downregulate the abnormal accumulation of the target α-synuclein associated with pathology of Parkinson's disease (PD).

In some embodiments, the pharmaceutical composition comprises an exosome—cationic matrix and a plasmid DNA. In some embodiments, the pharmaceutical composition comprises an exosome—cationic matrix and plasmid DNA and folic acid. In some embodiments, the pharmaceutical composition comprises an exosome—cationic matrix and plasmid DNA and rabies virus glycoprotein. In some embodiments, the pharmaceutical composition comprises an exosome—cationic matrix and plasmid DNA and rabies virus glycoprotein and folic acid. In some embodiments, the plasmid DNA carries structural sequences of p53. In some embodiments, the plasmid DNA carrying structural sequences of p53 is delivered to the brain at a concentration effective to introduce the p53 gene in brain. In some embodiments, the plasmid DNA carries coding sequences for Cas9-mediated knockout of NFκB as a model gene.

In some embodiments, the pharmaceutical composition comprises an exosome—curcuminoid complex. In some embodiments, the pharmaceutical composition comprises an exosome—curcuminoid complex and rabies virus glycoprotein (RVG), folic acid (FA), or a combination thereof.

The present development is a method to mitigate neuroinflammatory diseases by delivering siRNA or curcuminoids to the brain wherein the siRNA or curcuminoids are administered via a topical trans-spinal route. In some embodiments, the method comprises administering via a topical trans-spinal route an exosome—cationic matrix and siRNA or an exosome—curcuminoid complex. In some embodiments, the method comprises administering via a topical trans-spinal route an exosome—cationic matrix and siRNA and folic acid, or an exosome—curcuminoid complex and RVG or FA or a combination thereof. In some embodiments, the method comprises administering via a topical trans-spinal route an exosome—cationic matrix and siRNA and rabies virus glycoprotein (RVG). In some embodiments, the method comprises administering via a topical trans-spinal route an exosome—cationic matrix and siRNA and rabies virus glycoprotein (RVG) and folic acid (FA). In some embodiments, the method comprises administering via a topical trans-spinal route an exosome—cationic matrix or an exosome—curcuminoid complex comprising a milk exosome or a colostrum exosome or a combination thereof. In some embodiments, the method comprises administering via a topical trans-spinal route an exosome-curcuminoid complex at a concentration effective to reduce the neuroinflammation to baseline levels. In some embodiments, the method comprises administering via a topical trans-spinal route an exosome-cationic matrix and siNFκB. In some embodiments, the method comprises administering via a topical trans-spinal route an exosome—cationic matrix and siNFκB at a concentration effective to reduce the inflammatory regulator NFκB to baseline levels.

### BRIEF DESCRIPTION OF THE FIGURES

FIG. 1 is a graph showing the brain accumulation of Cy7-labeled EPM-siRNA in mice wherein the siRNA is siNFκB, and the EPM-siRNA is delivered intravenously (i.v.) or via a topical trans-spinal route (t.s.), using non-functionalized exosomes (EPM), folic acid functionalized exosomes (FA-EPM), and rabies virus glycoprotein plus folic acid functionalized exosomes (RVG/FA-EPM);

FIG. 2 is an autoradiograph showing the biodistribution of FA-EPM-siNFκB and free siNFxB (using P-32 label siRNA) in WT mice treated trans-spinally (t.s.);

FIG. 3 is a set of images showing the brain accumulation of Cy7-labeled exosomes in mice wherein the labeled exosome complex is delivered intravenously (i.v.) or via a topical trans-spinal route (t.s.), using non-functionalized exosomes (Exo), and folic acid functionalized exosomes (FA-Exo), and rabies virus glycoprotein and folic acid functionalized exosomes (RVG/FA-Exo) with (A) showing representative ex vivo tissue images showing the distribution of labeled exosomes and (B) showing the quantification of fluorescent intensity in brain tissue;

FIG. 4 is a graph showing liver, lung and brain tissue accumulation of curcuminoids in mice wherein a folic acid functionalized exosome—curcuminoid (FA-ExoCUR/BDMC) complex is delivered orally or via a topical trans-spinal (t.s.) route; and,

FIG. 5 is an autoradiograph showing the biodistribution of folate receptor FRα in WT mice.

### DETAILED DESCRIPTION OF THE PRESENT DEVELOPMENT

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The present development is a pharmaceutical composition for delivering therapeutics selected from nucleic acids or curcuminoids to the brain. Specifically, the present development is a pharmaceutical composition for delivering siRNA, anti-sense oligomer (ASO), plasmid DNA, or curcuminoids, specifically curcumin (CUR or CUR-I), demethoxycurcumin (DMC or CUR-II) and bisdemethoxycurcumin (BDMC or CUR-III), to the brain wherein the siRNA, ASO, plasmid DNA or curcuminoids are administered via a topical trans-spinal route. In some embodiments, the composition comprises an exosome—cationic matrix, also referred to herein as EPM, or an exosome—curcuminoid complex, also referred to herein as ExoCUR. In some embodiments, the composition comprises a functionalized exosome—curcuminoid complex, also referred to herein as {Fx}-ExoCUR, where {Fx} designates the exosome functionalizing agent.

The exosomes used in the EPM or the ExoCUR can be milk exosomes or colostrum exosomes or a combination thereof. The milk exosomes or colostrum exosomes may be isolated from raw milk or from raw colostrum immediately before use in the exosome—cationic matrix or the exosome-curcuminoid complex, or previously isolated exosomes may be used, including exosomes that have been isolated and frozen or exosomes that are in powdered form. In an exemplary embodiment, exosomes isolated from colostrum powder and having an average size less than 85 nm are used for the EPM complex or the ExoCUR complex. In an exemplary embodiment, exosomes isolated from colostrum powder and having an average size less than about 68 nm are used for the EPM complex and exosomes isolated from colostrum powder and having an average size less than about 69 nm are used for the ExoCUR complex. In an exemplary embodiment, exosomes isolated from colostrum powder and having an average size 45±3.6 nm, a PDI 0.241±0.03 and ZP in water −27±0.15 mV, are used for the EPM complex, and exosomes isolated from colostrum exosomes isolated from colostrum powder and having an average size 66±2.5 nm, a PDI 0.27±0.01 and ZP in water −9.2±0.70 mV, are used for the ExoCUR complex.

In some embodiments, the exosome is functionalized with rabies virus glycoprotein (RVG). In some embodiments, the exosome is functionalized with folic acid (FA). In some embodiments, the exosome is functionalized with RVG and FA. Without being bound by theory, it is believed that RVG assists nanocarriers in traversing the blood brain barrier (BBB). Once across the BBB, FA facilitates delivery to the cortex and hippocampus. Functionalizing exosomes with FA is believed to target the FA cognate receptors found in higher abundance in key regions of the brain, such as the choroid plexus folate receptor-α (FRα).

The following examples are provided herein for illustrative purposes only and not to limit the scope of the invention in any way.

Example 1—General procedure for preparation of the EPM complex: The milk exosomes or colostrum exosomes are incubated with a preselected polycation to form the EPM. The polycation may be any polycation known in the art for transfection or that can serve as a vehicle for delivery of nucleic acids, such as but not limited to polyethylenimine, polyethylenimine conjugates, polycationic peptides, polylysine, polyornithine, polyhistidine, polyarginine, DEAE-dextran, chitosan, polyamine dendrimers, cationic lipids, cationic phospholipids, and combinations thereof. In a preferred embodiment, the polycation is polyethylenimine.

Example 2—General procedure for preparation of the EPM-nucleic acid complex: The EPM is incubated with a nucleic acid to form an EPM-nucleic acid complex. In some embodiments, the nucleic acid is an siRNA, thereby forming an EPM-siRNA composition. In some embodiments, the siRNA is siNFκB or an siRNA targeting against NFκB expression. In some embodiments, the siRNA is siBACE1, siSTAT3 or siEGFR. Compositions or complexes comprising EPM and siRNA may be referred to herein as EPM-siRNA or EPM-siRNA complexes. Methods of preparing EPM-siRNA compositions generally are disclosed in PCT/US20/15259 filed 27 Jan. 2020, which is incorporated herein in its entirety by reference. In some embodiments, the nucleic acid is an ASO, thereby forming an EPM-ASO composition. In some embodiments, the ASO is ASO-α-synuclein. Compositions or complexes comprising EPM and ASO may be referred to herein as EPM-ASO or EPM-ASO complexes. In some embodiments, the nucleic acid is a plasmid DNA (pDNA), thereby forming an EPM-pDNA composition. In some embodiments, the plasmid DNA carries structural sequences of p53. The resulting EPM-nucleic acid complex is a liquid.

Example 3—General procedure for ExoCUR/BDMC complex: Milk exosomes or colostrum exosomes are incubated with a mixture of CUR and BDMC (exosomes: drug, 2:1, w/w) in the presence of ethanol in PBS. Unbound curcuminoids are removed, such as by low-speed centrifugation, and the exosomal formulation is collected, such as by ultracentrifugation or ultrafiltration, suspended in PBS and stored at −80° C. In an exemplary embodiment, the composition is an ExoCUR/BDMC complex comprising a total curcuminoid load of 60% CUR and 39% BDMC, and having an average size 136 nm, PDI 0.334 and ZP-12.8. The resulting ExoCUR/BDMC complex is a liquid.

Example 4—General procedure for ExoCUR complex: ExoCUR samples are prepared using exosomes isolated from bovine colostrum, and curcuminoids dissolved in ethanol:acetonitrile (1:1) or ethanol at 20 mg/ml. Both exosomes and curcuminoids were present at 1 ug/ul, except wherever indicated. The exosomes are incubated with either curcumin, or individual curcuminoids, or a mixture of different curcuminoids in predetermined ratios. After incubation, the free curcuminoids are removed, such as via centrifuge, and the supernatant is retained. The crude ExoCUR sample is isolated from the supernatant as a solid material, then resuspended, homogenized, and purified to remove any remaining free curcuminoids and/or coagulated material. Exemplary curcuminoid loadings are shown in Tables 1-3.

TABLE 1

| | % Load of | | | |
|---|---|---|---|---|
| CUR:CUR-III | CUR-I | CUR-II | CUR-III | Total Drug (%) |
| CUR | 42 | 10 | 2 | 54 |
| CUR:CUR-III, 2:1 | 39 | 8 | 33 | 81 |
| CUR-I:CUR-III, 2:1 | 48 | 0 | 28 | 76 |
| CUR-II:CUR-III, 2:1 | 0 | 54 | 30 | 85 |
| CUR:CUR-III, 1:2 | 0 | 1 | 2 | 3 |

TABLE 2

| | % Load of | | | |
|---|---|---|---|---|
| CUR:CUR-III | CUR-I | CUR-II | CUR-III | Total Drug (%) |
| CUR | 40 | 10 | 3 | 53 |
| CUR-I | 14 | 0 | <1 | 15 |
| CUR-II | 0 | 84 | <1 | 84 |
| CUR:CUR-III, 2:1 | 41 | 8 | 35 | 84 |
| CUR-I:CUR-III, 2:1 | 27 | 0 | 41 | 68 |
| CUR-II:CUR-III, 2:1 | <1 | 57 | 33 | 90 |

TABLE 3

| CUR:CUR-III | Total Drug Load(%) |
|---|---|
| CUR-II:CUR-III, 2:1 | 64 |
| CUR-II:CUR-III, 1.2:1 | 38 |
| CUR-II:CUR-III, 0.82:1 | 8 |

Example 5—General procedure for ExoCUR-III complex: ExoCUR-III samples are prepared using exosomes isolated from bovine colostrum, and CUR-III dissolved in ethanol:acetonitrile (1:1) or ethanol at 20 mg/ml. The exosomes are incubated with bisdemethoxycurcumin (BDMC or CUR-III) and 2-hydroxypropyl-beta-cyclodextrin (HPCD) in predetermined ratios. After incubation, unbound CUR-III and HPCD is removed using ultrafiltration. In an exemplary embodiment, unbound CUR-III and HPCD is removed using ultrafiltration with a spin filer of 30,000 molecular weight cut-off. Table 4 summarizes the loading of CUR-III in the presence and absence of HPCD.

TABLE 4

| Exo (ug/ul) | Exo:CUR-III | HPCD | Loading (%) |
|---|---|---|---|
| 2 | 2:1 | 0% | 0.5% |
| 1 | 2:1 | 0% | 1.5% |
| 2 | 2:1 | 4% | 6.5% |
| 2 | 2:1 | 5% | 12.6% |
| 2 | 2:1 | 6% | 29% |
| 2 | 2:1 | 6% | 33.3% |
| 2 | 2:1 | 8% | 30.8% |
| 2 | 2:1 | 10% | 19% |

In some embodiments, the EPM-nucleic acid complex or the ExoCUR complex, including ExoCUR/BDMC and ExoCUR-III, forms part of a pharmaceutical composition, wherein the EPM-nucleic acid complex or the ExoCUR complex is combined with a pharmaceutically-acceptable vehicle, carrier, or excipient. The pharmaceutically-acceptable carrier may be aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. For topical application, the EPM-nucleic acid complex or the ExoCUR complex can be combined with an ointment, a cream formulation, a pharmaceutical cream base, or a combination thereof. Optionally, the ointment, cream formulation, pharmaceutical cream base, or combination thereof, can further include active ingredients and various supports and vehicles. In some embodiments, the EPM-nucleic acid complex or the ExoCUR complex, either with or without a carrier, can be delivered using a topical patch or similar means that will allow the EPM-nucleic acid complex to be held in direct contact with the recipient's skin.

The EPM-nucleic acid complex is administered via a topical trans-spinal (t.s.) route. Trans-spinal delivery is a form of transdermal (t.d.) delivery that is applied specifically to the cervical/spinal area over the back of the neck. The t.s. route has several advantages over oral and i.v. routes, including reduced adverse effects, higher bioavailability, prolonged duration of action and reduced dosing frequency. Without being bound by theory, it is believed that t.s. route administration of the EPM-nucleic acid complex results in a higher concentration of siRNA delivered to the brain.

The EPM-siRNA complex is used in a method to mitigate neuroinflammatory diseases by administering the EPM-siRNA complex via the topical trans-spinal (t.s.) route. In some embodiments, the siRNA is siNFκB delivered to the brain at a concentration effective to reduce the inflammatory regulator NFκB to baseline levels. In some embodiments, the siRNA is siBACE1 delivered to the brain at a concentration effective to downregulate the overexpression of the target BACE1 (beta-site amyloid precursor protein cleaving enzyme 1) associated with AD pathology. In some embodiments, the siRNA is siSTAT3 delivered to the brain at a concentration effective to knockdown mutated STAT3 associated with the progression of glioblastomas. In some embodiments, the siRNA is siEGFR delivered to the brain at a concentration effective to downregulate the amplification and overexpression of the target epidermal growth factor receptor (EGFR) associated with glioblastomas.

The EPM-ASO complex is used in a method to downregulate gene expression by administering the EPM-ASO complex via the topical trans-spinal (t.s.) route. In some embodiments, the ASO is ASO-α-synuclein delivered to the brain at a concentration effective to downregulate the abnormal accumulation of the target α-synuclein associated with pathology of Parkinson's disease (PD).

The ExoCUR/BDMC complex is used in a method to treat and/or mitigate the progression of neuroinflammatory diseases, such as AD, by administering the ExoCUR/BDMC complex via a topical trans-spinal (t.s.) route. Trans-spinal delivery is a form of transdermal (t.d.) delivery that is applied specifically to the cervical/spinal area over the back of the neck. The t.s. route has several advantages over oral and i.v. routes, including reduced adverse effects, higher bioavailability, prolonged duration of action and reduced dosing frequency. Without being bound by theory, it is believed that t.s. route administration of the ExoCUR/BDMC complex results in a higher concentration of curcuminoids being delivered to the brain.

In some embodiments, the method comprises administering via the t.s. route the EPM-nucleic acid complex or the ExoCUR complex or a combination thereof. In some embodiments, the method comprises administering via the t.s. route the EPM-nucleic acid complex or the ExoCUR complex, or a combination thereof, wherein the exosome is selected from a milk exosome or a colostrum exosome or a combination thereof. In some embodiments, the method comprises administering via the t.s. route the EPM-nucleic acid complex or the ExoCUR complex, or a combination thereof, wherein the exosome is functionalized with FA, with RVG, or with a combination thereof. In some embodiments, the method comprises administering via the t.s. route the EPM-nucleic acid complex wherein the EPM-nucleic acid complex is the EPM-siRNA complex, or the EPM-ASO complex, or the EPM-pDNA complex. In some embodiments, the method comprises administering via the t.s. route the EPM-siRNA complex wherein the siRNA is siNFκB, SiBACE1, siSTAT3 or siEGFR. In some embodiments, the method comprises administering via the t.s. route the EPM-ASO complex wherein the ASO is ASO-α-synuclein. In some embodiments, the method comprises administering via the t.s. route the ExoCUR complex wherein the ExoCUR complex is the ExoCUR/BDMC complex, or the ExoCUR-III complex, or a combination thereof.

In some embodiments, the method comprises administering via the t.s. route the EPM-siRNA complex wherein the siRNA is siNFκB at a concentration effective to reduce the inflammatory regulator NFκB to baseline levels. As used herein, “baseline level” is defined as the concentration of NFκB found in the brain when no measurable inflammation is present. Optionally, the EPM-siRNA complex can be combined with a pharmaceutically acceptable vehicle, carrier, or excipient to form part of a pharmaceutical composition prior to administration. In an exemplary embodiment, the EPM-siRNA complex, either with or without a carrier, can be delivered using a topical patch or similar means that will allow the EPM-siRNA complex to be held in direct contact with the recipient's skin.

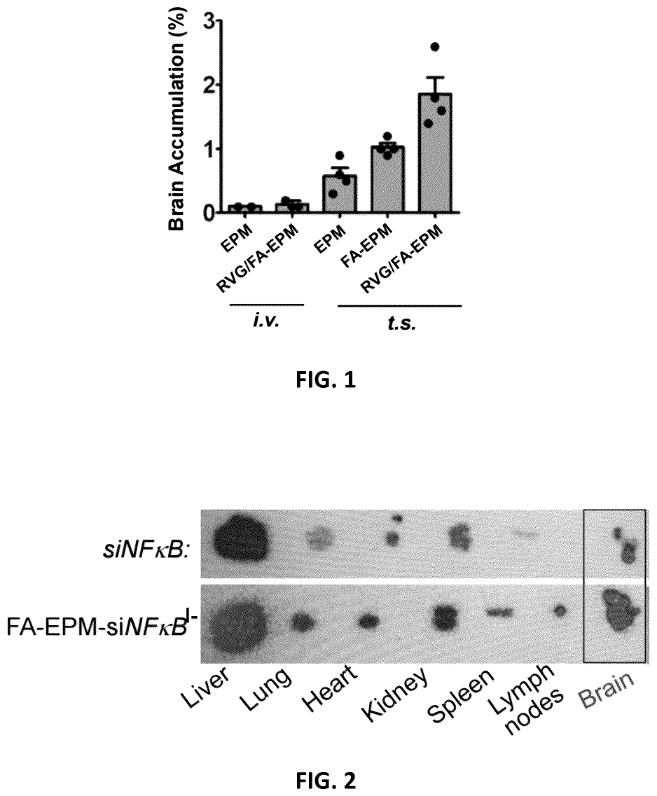

It has been found that using the EPM, siRNA entrapment of greater than 90% can be achieved. The EPM-siRNA complex demonstrates significantly less enzymatic degradation than free or uncomplexed siRNA. Likewise, using the EPM, ASO entrapment of greater than 90% can be achieved. The EPM-siNFκB provides higher knockdown of NFκB in murine fibroblast L929 cells than conventional reagents. As shown in FIG. 1, EPM-siRNA complexes comprising exosomes functionalized with FA have enhanced brain accumulation due to high expression of folate receptor-α (FRα) relative to EPM-siRNA complexes comprising exosomes without FA, and EPM-siRNA complexes comprising exosomes functionalization with RVG alone or together with FA have enhanced brain accumulation relative to EPM-siRNA complexes comprising exosomes without RVG. Further, as shown in FIG. 1, when the EPM-siRNA complex is administered via t.s. route there is a significantly higher percent brain accumulation of siRNA than when the EPM-siRNA complex is administered via i.v. route. For example, the percent brain accumulation of EPM-siNFκB formulations via t.s. administration was 10-15-times higher than by the i.v. administration. As shown in FIG. 2, it has also been found that that t.s. administered FA-EPM-siNFκB delivers siRNA 5- to 6-fold higher compared with free siNFκB t.s. administration to the brain in studies using WT mice.

In some embodiments, the method comprises administering via the t.s. route the ExoCUR/BDMC complex wherein the curcuminoids are at a concentration effective to reduce the inflammation level in the brain to baseline levels. As used herein, “baseline level” is defined as the concentration of curcuminoids found in the brain when aberrant AD-related gene expression is ameliorated, AD biomarkers are returned to pre-disease levels and improved cognitive function due to synergistic activity via different mechanisms is observed. Optionally, the ExoCUR/BDMC complex can be combined with a pharmaceutically acceptable vehicle, carrier, or excipient to form part of a pharmaceutical composition prior to administration. In an exemplary embodiment, the ExoCUR/BDMC complex, either with or without a carrier, can be delivered using a topical patch or similar means that will allow the ExoCUR/BDMC complex to be held in direct contact with the recipient's skin.

In some embodiments, the ExoCUR or ExoCUR/BDMC formulation administered by topical applications can be used for skin diseases, such as exzema, as Cy7-labeled exosomes were found in significant amounts in targeted local skin and muscle tissue.

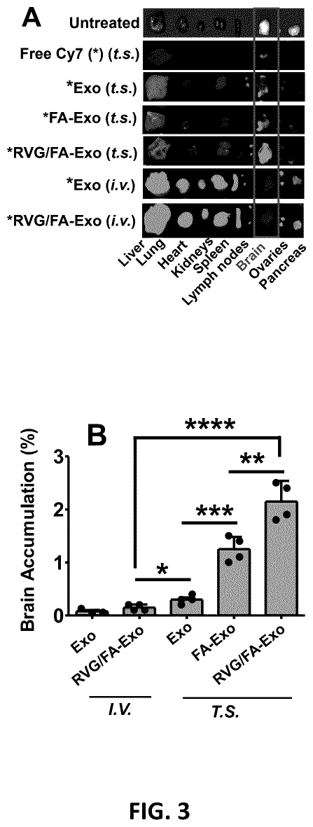

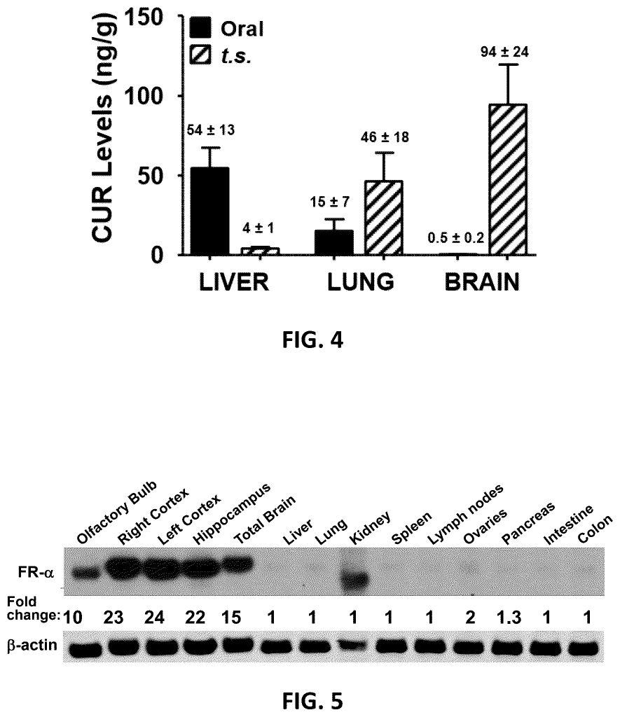

As shown in FIG. 3, exosomes functionalized with FA or with FA and RVG have enhanced brain accumulation relative to exosomes without FA or RVG, and functionalized exosomes have enhanced brain accumulation relative to non-functionalized exosomes. Further, as shown in FIG. 3, when the functionalized exosomes are administered via the t.s. route there is a significantly higher percent brain accumulation of exosomes than when the exosomes are administered via the i.v. route. As shown in FIG. 4, the t.s. administered FA-ExoCUR/BDMC complex delivers a significantly higher concentration of curcuminoids to the brain than are delivered to the liver or lungs. Further as shown in FIG. 4, t.s. administration of the FA-ExoCUR/BDMC complex is effective for delivery of curcuminoids to the brain whereas oral administration of the FA-ExoCUR/BDMC complex is effective for delivery of curcuminoids to the liver. As shown in FIG. 5, it has also been found that that t.s. administered FA-ExoCUR/BDMC complex results in higher drug accumulation in the brain due to high FRα expression in the cortex and hippocampus of the brain in studies using WT mice.

Unless defined otherwise, all technical and scientific terms used herein have the same meaning as commonly understood by one of ordinary skill in the art to which the presently disclosed subject matter pertains. All patents, patent applications, published applications and publications, databases, websites and other published materials referred to throughout the entire disclosure herein, unless noted otherwise, are incorporated by reference in their entirety. Representative methods, devices, and materials are described herein, but are not intended to be limiting unless so noted.

The terms “a”, “an”, and “the” refer to “one or more” when used in the subject specification, including the claims. Thus, for example, reference to “an exosome” includes a plurality of such exosomes, and so forth. The term “ambient temperature” as used herein refers to an environmental temperature of from about 0° F. to about 120° F., inclusive.

Unless otherwise indicated, all numbers expressing quantities of components, conditions, and otherwise used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, or percentage can encompass variations of, in some embodiments ±20%, in some embodiments ±10%, in some embodiments ±5%, in some embodiments ±1%, in some embodiments ±0.5%, and in some embodiments to ±0.1%, from the specified amount, as such variations are appropriate in the disclosed application.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All compositional percentages used herein are presented on a “by weight” basis, unless designated otherwise.

It is understood that, in light of a reading of the foregoing description, those with ordinary skill in the art will be able to make changes and modifications to the present invention without departing from the spirit or scope of the invention, as defined herein. For example, those skilled in the art may substitute materials supplied by different manufacturers than specified herein without altering the scope of the present invention.

What is claimed is:

1. A method to mitigate neuroinflammatory diseases comprising administering via a topical trans-spinal route at least one therapeutic agent, wherein the therapeutic agent is at least one nucleic acid or at least one curcuminoid or a combination thereof, and wherein the therapeutic agent is delivered using a composition comprising (a) an exosome—cationic matrix and a nucleic acid (“EPM-nucleic acid”), or (b) an exosome and a curcuminoid (“ExoCUR”).

2. The method of claim 1 wherein the therapeutic is siRNA, anti-sense oligomer (ASO), plasmid DNA, a curcuminoid, curcumin, demethoxycurcumin, bisdemethoxycurcumin, or a combination thereof.

3. The composition of claim 2 wherein the siRNA is siNFκB, siBACE1, siSTAT3 or siEGFR.

4. The composition of claim 2 wherein the ASO is ASO-α-synuclein.

5. The method of claim 1 wherein the exosome is a milk exosome or a colostrum exosome or a combination thereof.

6. The method of claim 5 wherein the exosome is functionalized with folic acid, rabies virus glycoprotein, or a combination thereof.

7. The method of claim 1 wherein the exosome—cationic matrix comprises polyethylenimine, polyethylenimine conjugates, polycationic peptides, polylysine, polyornithine, polyhistidine, polyarginine, DEAE-dextran, chitosan, polyamine dendrimers, cationic lipids, cationic phospholipids, and combinations thereof.

8. The method of claim 3 wherein the siNFκB is delivered to the brain at a concentration effective to reduce the inflammatory regulator NFκB to baseline levels.

9. The method of claim 3 wherein the siBACE1 is delivered to the brain at a concentration effective to downregulate the overexpression of the target BACE1 (beta-site amyloid precursor protein cleaving enzyme 1) associated with AD pathology.

10. The method of claim 3 wherein the siSTAT3 is delivered to the brain at a concentration effective to knockdown STAT3 associated with the progression of glioblastomas.

11. The method of claim 3 wherein the siEGFR delivered to the brain at a concentration effective to downregulate the amplification and overexpression of the target epidermal growth factor receptor (EGFR) associated with glioblastomas.

12. The method of claim 4 wherein the ASO-α-synuclein delivered to the brain at a concentration effective to downregulate the abnormal accumulation of the target α-synuclein associated with pathology of Parkinson's disease (PD).

13. The method of claim 1 wherein the ExoCUR complex is delivered to the brain at a concentration effective to reduce the inflammation.

14. The method of claim 1 wherein the complex is blended with a pharmaceutically acceptable vehicle, carrier, or excipient.

15. The method of claim 1 wherein the composition is administered using a patch.

16. A pharmaceutical composition for delivering therapeutics to the brain wherein the therapeutic is a nucleic acid or a curcuminoid or a combination thereof, and wherein the therapeutic is administered via a topical trans-spinal route, and wherein the composition comprising an exosome—cationic matrix plus nucleic acid complex (“EPM-nucleic acid complex”) or an exosome—curcuminoid complex (“ExoCUR”).

17. The composition of claim 16 wherein the therapeutic is siRNA, anti-sense oligomer (ASO), plasmid DNA, a curcuminoid, curcumin, demethoxycurcumin, bisdemethoxycurcumin, or a combination thereof.

18. The composition of claim 16 wherein the exosome is a milk exosome or a colostrum exosome or a combination thereof.

19. The composition of claim 16 further comprising a pharmaceutically acceptable vehicle, carrier, or excipient.

20. The method of claim 16 wherein the composition is administered using a patch.
